SEQUENCE LISTING (26 sequences)

SEQ ID NO: 1 - DNA, 58 bp, Homo sapiens
atgaagtggtgctggggcccagtgctgctcatcgcgggtgccacagtcctcatggagg

SEQ ID NO: 2 - protein, 20 aa, Homo sapiens
MKWCWGPVLLIAGATVLMEG

SEQ ID NO: 3 - DNA, 21 bp, Homo sapiens
gtcttcaagccgctcagcgtg

SEQ ID NO: 4 - protein, 7 aa, Homo sapiens
LQAAQRA

SEQ ID NO: 5 - DNA, 163 bp, Homo sapiens
cctgtggacagcgtggccccggcccccccaagcctcaggagggcaacacagtccctggcgagtggccctggcaggccagtgtgaggaggcaaggagcccacatctgcagcggctccctggtggcagacacctgggtcctcactgctgcccactgctttgaaaa

SEQ ID NO: 6 - protein, 54 aa, Homo sapiens
CGQRGPGPPKPQEGNTVPGEWPWQASVRRQGAHICSGSLVADTWVLTAAHCFEK

SEQ ID NO: 7 - DNA, 266 bp, Homo sapiens
ggcagcagcaacagaactgaattcctggtcagtggtcctgggttctctgcagcgtgagggactcagccctggggccgaagaggtgggggtggctgccctgcagttgcccagggcctataaccactacagccagggctcagacctggccctgctgcagctcgcccaccccacgacccacacacccctctgcctgccccagcccgcccatcgcttcccctttggagcctcctgctgggccactggctgggatcaggacaccagtgatg

SEQ ID NO: 8 - protein, 89 aa, Homo sapiens
AAATELNSWSVVLGSLQREGLSPGAEEVGVAALQLPRAYNHYSQGSDLALLQLAHPTTHTPLCLPQPAHRFPFGASCWATGWDQDTSDA

SEQ ID NO: 9 - DNA, 155 bp, Homo sapiens
ctcctgggaccctacgcaatctgcgcctgcgtctcatcagtcgccccacatgtaactgtatctacaaccagctgcaccagcgacacctgtccaacccggcccggcctgggatgctatgtgggggcccccagcctggggtgcagggcccctgtcag

SEQ ID NO: 10 - protein, 51 aa, Homo sapiens
PGTLRNLRLRLISRPTCNCIYNQLHQRHLSNPARPGMLCGGPQPGVQGPCQ

SEQ ID NO: 11 - DNA, 220 bp, Homo sapiens
ggagattccgggggccctgtgctgtgcctcgagcctgacggacactgggttcaggctggcatcatcagctttgcatcaagctgtgcccaggaggacgctcctgtgctgctgaccaacacagctgctcacagttcctggctgcaggctcgagttcagggggcagctttcctggcccagagcccagagaccccggagatgagtgatgaggacagctgtgtag

SEQ ID NO: 12 - protein, 74 aa, Homo sapiens
GDSGGPVLCLEPDGHWVQAGIISFASSCAQEDAPVLLTNTAAHSSWLQARVQGAAFLAQSPETPEMSDEDSCVA

SEQ ID NO: 13 - DNA, 151 bp, Homo sapiens
cctgtggatccttgaggacagcaggtccccaggcaggagcaccctccccatggccctgggaggccaggctgatgcaccagggacagctggcctgtggcggagccctggtgtcagaggaggcggtgctaactgctgcccactgcttcattgg

SEQ ID NO: 14 - protein, 50 aa, Homo sapiens
CGSLRTAGPQAGAPSPWPWEARLMHQGQLACGGALVSEEAVLTAAHCFIG

SEQ ID NO: 15 - DNA, 245 bp, Homo sapiens
gcgccaggccccagaggaatggagcgtagggctggggaccagaccggaggagtggggcctgaagcagctcatcctgcatggagcctacacccaccctgaggggggctacgacatggccctcctgctgctggcccagcctgtgacactgggagccagcctgcggcccctctgcctgccctatcctgaccaccacctgcctgatggggagcgtggctgggttctgggacgggcccgcccaggagcag

SEQ ID NO: 16 - protein, 82 aa, Homo sapiens
RQAPEEWSVGLGTRPEEWGLKQLILHGAYTHPEGGYDMALLLLAQPVTLGASLRPLCLPYPDHHLPDGERGWVLGRARPGAG

SEQ ID NO: 17 - DNA, 146 bp, Homo sapiens
gcatcagctccctccagacagtgcccgtgaccctcctggggcctagggcctgcagccggctgcatgcagctcctgggggtgatggcagccctattctgccggggatggtgtgtaccagtgctgtgggtgagctgcccagctgtgag

SEQ ID NO: 18 - protein, 48 aa, Homo sapiens
ISSLQTVPVTLLGPRACSRLHAAPGGDGSPILPGMVCTSAVGELPSCE

SEQ ID NO: 19 - DNA, 276 bp, Homo sapiens
ggcctgtctggggcaccactggtgcatgaggtgaggggcacatggttcctggccgggctgcacagcttcggagatgcttgccaaggccccgccaggccggcggtcttcaccgcgctccctgcctatgaggactgggtcagcagtttggactggcaggtctacttcgccgaggaaccagagcccgaggctgagcctggaagctgcctggccaacataagtatgtggccccggggcctcctgccaaaccctgcctctccaggacccttctctctccag

SEQ ID NO: 20 - protein, 92 aa, Homo sapiens
GLSGAPLVHEVRGTWFLAGLHSFGDACQGPARPAVFTALPAYEDWVSSLDWQVYFAEEPEPEAEPGSCLANISMWPRGLLPNPASPGPFSLQ

SEQ ID NO: 21 - DNA, 1701 bp, Homo sapiens
atgaagtggtgctggggcccagtgctgctcatcgcgggtgccacagtcctcatggagggtcttcaagccgctcagcgtgcctgtggacagcgtggccccggcccccccaagcctcaggagggcaacacagtccctggcgagtggccctggcaggccagtgtgaggaggcaaggagcccacatctgcagcggctccctggtggcagacacctgggtcctcactgctgcccactgctttgaaaaggcagcagcaacagaactgaattcctggtcagtggtcctgggttctctgcagcgtgagggactcagccctggggccgaagaggtgggggtggctgccctgcagttgcccagggcctataaccactacagccagggctcagacctggccctgctgcagctcgcccaccccacgacccacacacccctctgcctgccccagcccgcccatcgcttcccctttggagcctcctgctgggccactggctgggatcaggacaccagtgatgctcctgggaccctacgcaatctgcgcctgcgtctcatcagtcgccccacatgtaactgtatctacaaccagctgcaccagcgacacctgtccaacccggcccggcctgggatgctatgtgggggcccccagcctggggtgcagggcccctgtcagggagattccgggggccctgtgctgtgcctcgagcctgacggacactgggttcaggctggcatcatcagctttgcatcaagctgtgcccaggaggacgctcctgtgctgctgaccaacacagctgctcacagttcctggctgcaggctcgagttcagggggcagctttcctggcccagagcccagagaccccggagatgagtgatgaggacagctgtgtagcctgtggatccttgaggacagcaggtccccaggcaggagcaccctccccatggccctgggaggccaggctgatgcaccagggacagctggcctgtggcggagccctggtgtcagaggaggcggtgctaactgctgcccactgcttcattgggcgccaggccccagaggaatggagcgtagggctggggaccagaccggaggagtggggcctgaagcagctcatcctgcatggagcctacacccaccctgaggggggctacgacatggccctcctgctgctggcccagcctgtgacactgggagccagcctgcggcccctctgcctgccctatcctgaccaccacctgcctgatggggagcgtggctgggttctgggacgggcccgcccaggagcaggcatcagctccctccagacagtgcccgtgaccctcctggggcctagggcctgcagccggctgcatgcagctcctgggggtgatggcagccctattctgccggggatggtgtgtaccagtgctgtgggtgagctgcccagctgtgagggcctgtctggggcaccactggtgcatgaggtgaggggcacatggttcctggccgggctgcacagcttcggagatgcttgccaaggccccgccaggccggcggtcttcaccgcgctccctgcctatgaggactgggtcagcagtttggactggcaggtctacttcgccgaggaaccagagcccgaggctgagcctggaagctgcctggccaacataagtatgtggccccggggcctcctgccaaaccctgcctctccaggacccttctctctccag

SEQ ID NO: 22 - protein, 567 aa, Homo sapiens
MKWCWGPVLLIAGATVLMEGLQAAQRACGQRGPGPPKPQEGNTVPGEWPWQASVRRQGAHICSGSLVADTWVLTAAHCFEKAAATELNSWSVVLGSLQREGLSPGAEEVGVAALQLPRAYNHYSQGSDLALLQLAHPTTHTPLCLPQPAHRFPFGASCWATGWDQDTSDAPGTLRNLRLRLISRPTCNCIYNQLHQRHLSNPARPGMLCGGPQPGVQGPCQGDSGGPVLCLEPDGHWVQAGIISFASSCAQEDAPVLLTNTAAHSSWLQARVQGAAFLAQSPETPEMSDEDSCVACGSLRTAGPQAGAPSPWPWEARLMHQGQLACGGALVSEEAVLTAAHCFIGRQAPEEWSVGLGTRPEEWGLKQLILHGAYTHPEGGYDMALLLLAQPVTLGASLRPLCLPYPDHHLPDGERGWVLGRARPGAGISSLQTVPVTLLGPRACSRLHAAPGGDGSPILPGMVCTSAVGELPSCEGLSGAPLVHEVRGTWFLAGLHSFGDACQGPARPAVFTALPAYEDWVSSLDWQVYFAEEPEPEAEPGSCLANISMWPRGLLPNPASPGPFSLQ

SEQ ID NO: 23 - DNA, 10 bp, Homo sapiens
gctcagcgtg

SEQ ID NO: 24 - protein, 4 aa, Homo sapiens
AQRA

SEQ ID NO: 25 - DNA, 1632 bp, Homo sapiens
gctcagcgtgcctgtggacagcgtggccccggcccccccaagcctcaggagggcaacacagtccctggcgagtggccctggcaggccagtgtgaggaggcaaggagcccacatctgcagcggctccctggtggcagacacctgggtcctcactgctgcccactgctttgaaaaggcagcagcaacagaactgaattcctggtcagtggtcctgggttctctgcagcgtgagggactcagccctggggccgaagaggtgggggtggctgccctgcagttgcccagggcctataaccactacagccagggctcagacctggccctgctgcagctcgcccaccccacgacccacacacccctctgcctgccccagcccgcccatcgcttcccctttggagcctcctgctgggccactggctgggatcaggacaccagtgatgctcctgggaccctacgcaatctgcgcctgcgtctcatcagtcgccccacatgtaactgtatctacaaccagctgcaccagcgacacctgtccaacccggcccggcctgggatgctatgtgggggcccccagcctggggtgcagggcccctgtcagggagattccgggggccctgtgctgtgcctcgagcctgacggacactgggttcaggctggcatcatcagctttgcatcaagctgtgcccaggaggacgctcctgtgctgctgaccaacacagctgctcacagttcctggctgcaggctcgagttcagggggcagctttcctggcccagagcccagagaccccggagatgagtgatgaggacagctgtgtagcctgtggatccttgaggacagcaggtccccaggcaggagcaccctccccatggccctgggaggccaggctgatgcaccagggacagctggcctgtggcggagccctggtgtcagaggaggcggtgctaactgctgcccactgcttcattgggcgccaggccccagaggaatggagcgtagggctggggaccagaccggaggagtggggcctgaagcagctcatcctgcatggagcctacacccaccctgaggggggctacgacatggccctcctgctgctggcccagcctgtgacactgggagccagcctgcggcccctctgcctgccctatcctgaccaccacctgcctgatggggagcgtggctgggttctgggacgggcccgcccaggagcaggcatcagctccctccagacagtgcccgtgaccctcctggggcctagggcctgcagccggctgcatgcagctcctgggggtgatggcagccctattctgccggggatggtgtgtaccagtgctgtgggtgagctgcccagctgtgagggcctgtctggggcaccactggtgcatgaggtgaggggcacatggttcctggccgggctgcacagcttcggagatgcttgccaaggccccgccaggccggcggtcttcaccgcgctccctgcctatgaggactgggtcagcagtttggactggcaggtctacttcgccgaggaaccagagcccgaggctgagcctggaagctgcctggccaacataagtatgtggccccggggcctcctgccaaaccctgcctctccaggacccttctctctccag

SEQ ID NO: 26 - protein, 544 aa, Homo sapiens
AQRACGQRGPGPPKPQEGNTVPGEWPWQASVRRQGAHICSGSLVADTWVLTAAHCFEKAAATELNSWSVVLGSLQREGLSPGAEEVGVAALQLPRAYNHYSQGSDLALLQLAHPTTHTPLCLPQPAHRFPFGASCWATGWDQDTSDAPGTLRNLRLRLISRPTCNCIYNQLHQRHLSNPARPGMLCGGPQPGVQGPCQGDSGGPVLCLEPDGHWVQAGTISFASSCAQEDAPVLLTNTAAHSSWLQARVQGAAFLAQSPETPEMSDEDSCVACGSLRTAGPQAGAPSPWPWEARLMHQGQLACGGALVSEEAVLTAAHCFIGRQAPEEWSVGLGTRPEEWGLKQLILHGAYTHPEGGYDMALLLLAQPVTLGASLRPLCLPYPDHHLPDGERGWVLGRARPGAGISSLQTVPVTLLGPRACSRLHAAPGGDGSPILPGMVCTSAVGELPSCEGLSGAPLVHEVRGTWFLAGLHSFGDACQGPARPAVFTALPAYEDWVSSLDWQVYFAEEPEPEAEPGSCLANISMWPRGLLPNPASPGPFSLQ

(19) United States
(12) Patent Application Publication
(10) Pub. No.: US 2006/0263776 A1
(43) Pub. Date: Nov. 23, 2006

(54) Serine protease

(76) Inventors: Richard Joseph Fagan, London (GB); David Michalovich, London (GB); Timothy Charles Oliver Nugent, London (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/538,197

(22) PCT Filed: Dec. 11, 2003
(86) PCT No.: PCT/GB03/05404

§ 371 Date: Dec. 9, 2005

(30) Foreign Application Priority Data
Dec. 11, 2002 (GB) 0228957.7
Dec. 11, 2002 (GB) 02289577

(51) Int. Cl. C12Q 1/68; C07H 21/04; A61K 38/54; A61K 48/00; C12N 9/64; C12P 21/06

(52) U.S. Cl. 435/6; 435/69.1; 435/320.1; 435/325; 514/12; 435/226; 536/23.2; 514/44

(57) ABSTRACT

This invention relates to a novel protein, termed INTP039, herein identified as a serine protease, in particular, as a member of the trypsin family of proteins and to the use of this protein and nucleic acid sequence from the encoding genes in the diagnosis, prevention, and treatment of disease.

This invention relates to a novel protein, termed INTP039, herein identified as a serine protease, in particular, as a member of the trypsin family of proteins and to the use of this protein and nucleic acid sequence from the encoding gene in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

### INTRODUCTION

Proteases are enzymes that irreversibly hydrolyse amide bonds in peptides and proteins. Proteases are widely distributed and are involved in many different biological processes, from activation of proteins and peptides to degradation of proteins. Despite the fact that proteases have been shown to be involved in many different diseases, drugs targeted to proteases are still rare in pharmacy, although inhibitors of angiotensin converting enzyme have been among the most successful antihypertensive drugs for several years. Proteases have recently received substantial publicity as valuable therapeutic targets following the approval of HIV protease inhibitors.

Proteases can be divided in large Families. The term “Family” is used to describe a group of proteases in which each member shows an evolutionary relationship to at least one other member, either throughout the whole sequence or at least in the part of the sequence responsible for catalytic activity. The name of each Family reflects the catalytic activity type of the proteases in the Family. Thus, serine proteases belong to the S family, threonine proteases belong to the T family, aspartyl proteases belong to the A family, cysteine proteases belong to the C family and metalloproteases belong to the M family. Certain proteases have an unknown mechanism of action and belong to the “U” family.

Serine Proteases (S Family)

The serine proteases are grouped into 7 clans, containing 39 families and over 750 different members from various species. Of these 39 families, only 9 contain members that are expressed in animals. Families with particularly interesting members include the S1 Family and the S26 Family. The S26 Family includes bacterial leader peptidase I, which has been implicated as an important agent in bacterial infection (Paetzel, M., et al., Proteins (1995), (1):122-5; Tschantz, W. R., Methods Enzymol. (1994), 244:285-301).

The S1 Family is the largest of these 9 families and the family with by far the greatest number of members of significance to health and disease. This family includes clotting factors such as factors VII, IX, X, XI and XII, proteases of the complement system, plasmin, trypsin, UPA, elastases such as leukocyte elastase, chymase, thrombin, cathepsin G, plasminogen, tryptase, urokinase and many others.

The prototypic protease of this family is chymotrypsin. The catalytic activity of the S1 family of proteases is provided by a charge relay system involving an aspartic acid residue that is hydrogen-bonded to a histidine, which itself is hydrogen-bonded to a serine. The sequences in the vicinity of the active site serine and histidine residues are well conserved in this family of proteases.

Serine proteases have been shown to play a role in diverse physiological functions, many of which can play a role in disease processes (see Table 1) such as cardiovascular disease (Kohler, H. T., et al., (2000) N. Engl. J. Med. 342(24):1792-801; Hamsten, A., et al., (2000) Thromb. Haemost. 83(3):397403; Califf, R. M., et al., (2000) Circulation, 101(19):2231-8; Krendel, S., et al., (2000) Ann. Emerg. Med. 35(5):502-5), cancer (Schmidt, M., et al., (1999) Acta Otolaryngol. 119(8):949-53; Raigoso, P., et al., (2000) Int. J. Biol. Markers, 15(l):44-50; Haese, A., et al., (2000) J. Urol. 163(5):1491-7; Hoopera, J. D., et al., (2000) Biochim. Biophys. Acta, 2000, 1492(l):63-71; Wallrapp, C., et al., (2000) Cancer Res. 2000, 60(10):2602-6; Cao, Y., et al., (2000) Int. J. Mol. Med. 2000, 5(5):547-51), asthma, chronic obstructive pulmonary disease (COPD), inflammatory diseases (Rice, K. D., et al., (1998) Curr. Pharm. Des., (5):381-96; Nadel, J. A., et al., (1998) Eur. Respir. J., (6):1250-1; Wright, C. D., et al., (1999) Biochem. Pharmacol., 58(12):1989-96; Burgess, L. E., et al., (1999) Proc. Natl. Acad. Sci. U. S. A., 96(15):8348-52; Barnes, P. J., et al., (2000) Chest, 117(2 Suppl):1OS-4S) and bacterial infections (Al-Hasani, K., et al., (2000) Infect. Immun., 68(5):2457-63; Gaillot, O., et al., (2000) Mol Microbiol. 35(6):1286-94; Lejal, N., et al., (2000) J. Gen. Virol., 81(Pt 4):983-92).

Alteration of their activity is a means to alter the disease phenotype and as such identification of novel serine proteases is highly relevant as they may play a role in the diseases identified as well as other disease states. As such, the identification of novel serine proteases is highly relevant for the treatment and diagnosis of disease, particularly those identified in Table 1 below.

TABLE 1

| | | Associated |
|---|---|---|
| Protease name | Biological function | disease |
| Thrombin | Converts fibrinogen to fibrin | Cardiovascular |
| Factor Xa | Converts prothrombin to thrombin in | Cardiovascular |
| | blood coagulation | |
| Factor VIIa | Proteolytically activates coagulation | Cardiovascular |
| | factors IX and X | |
| chymase | Hydrolyses extracellular matrix proteins | Asthma |
| | and others after release from mast cells | |
| tryptase | Hydrolyses extracellular matrix proteins | Asthma |
| | and others after release from mast cells | |
| Leukocyte | Proteolyses bacterial extracellular | Emphysema, |
| elastase | matrix components | cystic fibrosis, |
| | | adult |
| | | respiratory |
| | | distress |
| | | syndrome, |
| | | rheumatoid |
| | | arthritis |
| Thrombin | Clotting of blood by processing of | Cardiovascular |
| | fibrinogen | |
| Complement | Activation of complement | Xeno- |
| C1r and C1s | | transplantation, |
| | | sepsis |
| Urokinase | Activates plasminogen to plasmin in | Cardiovascular, |
| | fibrinolysis | oncology |
| Bacterial | Hydrolysis of signal peptides of | Bacterial |
| leader | secreted proteins other than lipoproteins | infections |
| peptidase I | | |

Increasing knowledge of these proteins is therefore of extreme importance in increasing the understanding of the underlying pathways that lead to the disease states and associated disease states mentioned above, and in developing more effective gene and/or drug therapies to treat these disorders.

### THE INVENTION

The invention is based on the discovery that the INTP039 polypeptide is a member of the trypsin family of proteins.

In one embodiment of the first aspect of the invention, there is provided a polypeptide which:
(i) comprises the amino acid sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 and/or SEQ ID NO:26;
(ii) is a fragment thereof which functions as a serine protease which is a member of the trypsin family of proteins, or has an antigenic determinant in common with the polypeptides of (i); or
(iii) is a functional equivalent of (i) or (ii).

Preferably, the polypeptide according to this first aspect of the invention:
(i) comprises the amino acid sequence as recited in SEQ ID NO:22 and/or SEQ ID NO:26;
(ii) is a fragment thereof which functions as a serine protease which is a member of the trypsin family of proteins, or has an antigenic determinant in common with the polypeptides of (i); or
(iii) is a functional equivalent of (i) or (ii).

According to a second embodiment of this first aspect of the invention, there is provided a polypeptide which:
(i) consists of the amino acid sequence as recited in SEQ ID NO:2, SEQ ID NO:4, SEQ ID NO:6, SEQ ID NO:8, SEQ ID NO:110, SEQ ID NO:12, SEQ ID NO:14, SEQ ID NO:16, SEQ ID NO:18, SEQ ID NO:20, SEQ ID NO:22, SEQ ID NO:24 and/or SEQ ID NO:26;
(ii) is a fragment thereof which functions as a serine protease which is a member of the trypsin family of proteins, or having an antigenic determinant in common with the polypeptides of (i); or
(iii) is a functional equivalent of (i) or (ii).

The polypeptide having the sequence recited in SEQ ID NO:2 is referred to hereafter as “INTP039 exon 1 polypeptide”. The polypeptide having the sequence recited in SEQ ID NO:4 is referred to hereafter as “INTP039 exon 2 polypeptide”. The polypeptide having the sequence recited in SEQ ID NO:6 is referred to hereafter as “INTP039 exon 3 polypeptide”. The polypeptide having the sequence recited in SEQ ID NO:8 is referred to hereafter as “INTP039 exon 4 polypeptide”. The polypeptide having the sequence recited in SEQ ID NO:10 is referred to hereafter as “INTP039 exon 5 polypeptide”. The polypeptide having the sequence recited in SEQ ID NO:12 is referred to hereafter as “INTP039 exon 6 polypeptide”. The polypeptide having the sequence recited in SEQ ID NO:14 is referred to hereafter as “INTP039 exon 7 polypeptide”. The polypeptide having the sequence recited in SEQ ID NO:16 is referred to hereafter as “INTP039 exon 8 polypeptide”. The polypeptide having the sequence recited in SEQ ID NO:18 is referred to hereafter as “INTP039 exon 9 polypeptide”. The polypeptide having the sequence recited in SEQ ID NO:20 is referred to hereafter as “INTP039 exon 10 polypeptide”. The polypeptide having the sequence recited in SEQ ID NO:22 is referred to hereafter as the “INTP039 polypeptide”.

Although the Applicant does not wish to be bound by this theory, it is postulated that the first 23 amino acids of the INTP039 polypeptide form a signal peptide.

The INTP039 exon 2 and full length INTP039 polypeptide sequences without this postulated signal sequence are recited in SEQ ID NO:24 and SEQ ID NO:26 respectively.

The polypeptide having the sequence recited in SEQ ID NO:24 is referred to hereafter as “the INTP039 exon 2 mature polypeptide”. The polypeptide having the sequence recited in SEQ ID NO:26 is referred to hereafter as “the INTP039 mature polypeptide”.

The term “INTP039 polypeptides” as used herein includes polypeptides comprising the INTP039 exon 1 polypeptide, the INTP039 exon 2 polypeptide, the INTP039 exon 3 polypeptide, the INTP039 exon 4 polypeptide, the INTP039 exon 5 polypeptide, the INTP039 exon 6 polypeptide, the INTP039 exon 7 polypeptide, the INTP039 exon 8 polypeptide, the INTP039 exon 9 polypeptide, the INTP039 exon 10 polypeptide, the INTP039 polypeptide, the INTP039 exon 2 mature polypeptide and the INTP039 mature polypeptide.

By “functions as a member of the trypsin family of proteins” we refer to polypeptides that comprise amino acid sequence or structural features that can be identified as conserved features within the polypeptides of the trypsin family, such that the polypeptide's interaction with ligand is not substantially affected detrimentally in comparison to the function of the full length wild type polypeptide. In particular, we refer to the presence of cysteine residues in specific positions within the polypeptide that allow the formation of intra-domain disulphide bonds. Protease activity can be detected by various assays including the ProCheck™ Universal Protease Assay (Serologicals Corporation). This is able to detect enzyme levels as low as 1×10⁻⁴ Units.

In a second aspect, the invention provides a purified nucleic acid molecule which encodes a polypeptide of the first aspect of the invention.

Preferably, the purified nucleic acid molecule comprises the nucleic acid sequence as recited in SEQ ID NO:1 (encoding the INTP039 exon 1 polypeptide), SEQ ID NO:3 (encoding the INTP039 exon 2 polypeptide), SEQ ID NO:5 (encoding the INTP039 exon 3 polypeptide), SEQ ID NO:7 (encoding the INTP039 exon 4 polypeptide), SEQ ID NO:9 (encoding the INTP039 exon 5 polypeptide), SEQ ID NO;11 (encoding the INTP039 exon 6 polypeptide), SEQ ID NO:13 (encoding the INTP039 exon 7 polypeptide), SEQ ID NO:15 (encoding the INTP039 exon 8 polypeptide), SEQ ID NO:17 (encoding the INTP039 exon 9 polypeptide), SEQ ID NO:19 (encoding the INTP039 exon 10 polypeptide), SEQ ID NO:21 (encoding the INTP039 polypeptide), SEQ ID NO:23 (encoding the INTP039 exon 2 mature polypeptide) and/or SEQ ID NO:25 (encoding the INTP039 mature polypeptide) or is a redundant equivalent or fragment of any one of these sequences.

The invention further provides that the purified nucleic acid molecule consists of the nucleic acid sequence as recited in SEQ ID NO;1 (encoding the INTP039 exon I polypeptide), SEQ ID NO:3 (encoding the INTP039 exon 2 polypeptide), SEQ ID NO:5 (encoding the INTP039 exon 3 polypeptide), SEQ ID NO:7 (encoding the INTP039 exon 4 polypeptide), SEQ ID NO:9 (encoding the INTP039 exon 5 polypeptide), SEQ ID NO:11 (encoding the INTP039 exon 6 polypeptide), SEQ ID NO;13 (encoding the INTP039 exon 7 polypeptide), SEQ ID NO:15 (encoding the INTP039 exon 8 polypeptide), SEQ ID NO:17 (encoding the INTP039 exon 9 polypeptide), SEQ ID NO:19 (encoding the INTP039 exon 10 polypeptide), SEQ ID NO:21 (encoding the INTP039 polypeptide), SEQ ID NO:23 (encoding the INTP039 exon 2 mature polypeptide) and/or SEQ ID NO:25 (encoding the INTP039 mature polypeptide) or is a redundant equivalent or fragment of any one of these sequences.

In a third aspect, the invention provides a purified nucleic acid molecule which hybridizes under high stringency conditions with a nucleic acid molecule of the second aspect of the invention.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to protein members of the trypsin family of the first aspect of the invention. Preferably, the ligand inhibits the function of a polypeptide of the first aspect of the invention which is a member of the trypsin family of proteins. Ligands to a polypeptide according to the invention may come in various forms, including natural or modified substrates, enzymes, receptors, small organic molecules such as small natural or synthetic organic molecules of up to 2000 Da, preferably 800 Da or less, peptidornimetics, inorganic molecules, peptides, polypeptides, antibodies, structural or functional mimetics of the aforementioned.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonise) the level of expression of the gene or the activity of the polypeptide.

Importantly, the identification of the function of the INTP039 polypeptides allows for the design of screening methods capable of identifying compounds that are effective in the treatment and/or diagnosis of disease. Ligands and compounds according to the sixth and seventh aspects of the invention may be identified using such methods. These methods are included as aspects of the present invention.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis of diseases in which members of the trypsin family are implicated. Such diseases may include cell proliferative disorders and/or differentiative disorders, such as carcinoma, sarcoma, solid tumours including prostate, breast, lung, testis, ovary, head and neck, brain or bone tumour, metastatic disorders or hematopoietic neoplastic disorders; disorders of bone metabolism, such as osteoporosis, osteodystrophy, osteomalacia, rickets, osteitis fibrosa cystica, renal osteodystrophy, osteosclerosis, osteopenia, fibrogenesis-imperfecta ossium, secondary hyperparathyrodism, hypoparathyroidism, hyperparathyroidism; periodontal disease, immune/autoimmune disorders such as, arthritis, multiple sclerosis, encephalomyelitis, myasthenia gravis, systemic lupus erythematosis, autoimmune thyroiditis, dermatitis, psoriasis, Sjogren's Syndrome, Crohn's disease, ulcer, iritis, conjunctivitis, ulcerative colitis, asthma, scleroderma, autoimmune uveitis, allergic encephalomyelitis, hearing loss, aplastic anemia, anemia, idiopathic thrombocytopenia, polychondritis, Graves' disease, graft-versus-host disease, allergy; hematopoietic disorders; cardiovascular disorders such as hypertension, atherosclerosis, congestive heart failure, coronary artery disease, arrhythmias, cardiomyopathies, artherosclerosis, hypertensive vascular disease, Raynaud disease, aneurysms, varicose veins, thrombophlebitis, phlebothrombosis, tumors, hemangioma, lymphangioma, glomus tumor (glomangioma), Kaposi sarcoma, angiosarcoma, hemangiopericytoma; blood clotting disorders, such as hemorrhagic diatheses, nonthrombocytopenic purpuras, thrombocytopenia, idiopathic thrombocytopenic purpura, HIV-associated thrombocytopenia, thrombotic microangiopathies, hemorrhagic diatheses, lymphomas, Hodgkin disease; liver disorders such as, cirrhosis, hepatocellular necrosis, liver fibrosis, Al-antitrypsin deficiency, hemochromatosis copper storage diseases and liver damage (especially drug or alcohol induced); bacterial, fungal or viral infection; pain or metabolic disorders, such as obesity, anorexia nervosa, cachexia, lipid disorders, and diabetes; AIDS and other pathological conditions.

These molecules may also be used in the manufacture of a medicament for the treatment of such diseases.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as a serine protease, particularly as a member of the trypsin family. Suitable uses of the polypeptides of the invention as trypsin proteins include use as a regulator of cellular growth, metabolism or differentiation, use as part of a receptor/ligand pair and use as a diagnostic marker for a physiological or pathological condition selected from the list given above.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro portion to produce an active mature polypeptide. In such polypeptides, the pre-, pro- or prepro-sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the INTP039 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the INTP039 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

Typically, greater than 30% identity between two polypeptides is considered to be an indication of functional equivalence. Preferably, functionally equivalent polypeptides of the first aspect of the invention have a degree of sequence identity with the INTP039 polypeptide, or with active fragments thereof, of greater than 80%. More preferred polypeptides have degrees of identity of greater than 85%, 90%, 95%, 98% or 99%, respectively.

The functionally-equivalent polypeptides of the first aspect of the invention may also be polypeptides which have been identified using one or more techniques of structural alignment. For example, the Inphannatica Genome Threader technology that forms one aspect of the search tools used to generate the Biopendium™ search database may be used (see PCT application WO 01/69507) to identify polypeptides of presently-unknown function which, while having low sequence identity as compared to the LNTP039 polypeptides, are predicted to be members of the trypsin family, by virtue of sharing significant structural homology with the INTP039 polypeptide sequence. By “significant structural homology” is meant that the Inpharmatica Genome Threader predicts two proteins to share structural homology with a certainty of 10% and above.

The polypeptides of the first aspect of the invention also include fragments of the INTP039 polypeptides and fragments of the functional equivalents of the INTP039 polypeptides, provided that those fragments are members of the trypsin family or have an antigenic determinant in common with the INTP039 polypeptides.

As used herein, the term “fragment” refers to a polypeptide having an amino acid sequence that is the same as part, but not all, of the amino acid sequence of the INTP039 polypeptide or one of their functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant.

Fragments of the full length INTP039 polypeptides may consist of combinations of 2, 3, 4, 5, 6, 7, 8, 9 or 10 of neighbouring exon sequences in the INTP039 polypeptide sequences, respectively. For example, such combinations include exons 1 and 2, 2 and 3 or 1, 2 and 3. Such fragments are included in the present invention.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as wilt be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the first aspect of the invention.

By “substantially greater affinity” we mean that there is a measurable increase in the affinity for a polypeptide of the invention as compared with the affinity for known secreted proteins.

Preferably, the affinity is at least 1.5-fold, 2-fold, 5-fold 10-fold, 100-fold, 10³-fold, 10⁴-fold, b 10⁵-fold, 10⁶-fold or greater for a polypeptide of the invention than for known secreted proteins such as members of the IL-8 chemokine family of proteins.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl Acad. Sci. USA, 86, 10029 (1989); Gorrnan et al., Proc. Natl Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode a polypeptide sequence as recited in SEQ ID NO:2, SEQ ID NO:4, SEQ ID NO:6, SEQ D NO:8, SEQ ID NO;10, SEQ ID NO;12, SEQ ID NO;14, SEQ ID NO;16, SEQ ID NO:18, SEQ ID NO:20, SEQ ID NO:22, SEQ ID NO:24 and SEQ ID NO:26 and functionally equivalent polypeptides. These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes a polypeptide of this invention may be identical to the coding sequence of one or more of the nucleic acid molecules disclosed herein.

These molecules also may have a different sequence which, as a result of the degeneracy of the genetic code, encodes a polypeptide SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:26. Such nucleic acid molecules may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention. Such a nucleic acid molecule may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (199 1); Lee et al., Nucleic Acids Res 6, 3073 (1979); Cooney et al., Science 241, 456 (1988); Dervan et al., Science 251, 1360 (1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al. [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5× Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to a nucleic acid molecule encoding the INTP039 polypeptides and nucleic acid molecules that are substantially complementary to such nucleic acid molecules. Preferably, a nucleic acid molecule according to this aspect of the invention comprises a region that is at least 80% identical over its entire length to such coding sequences, or is a nucleic acid molecule that is complementary thereto. In this regard, nucleic acid molecules at least 90%, preferably at least 95%, more preferably at least 98%, 99% or more identical over their entire length to the same are particularly preferred. Preferred embodiments in this respect are nucleic acid molecules that encode polypeptides which retain substantially the same biological function or activity as the INTP039 polypeptides.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INTP039 polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INTP039 polypeptide is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989,1992). Probes comprising at least 15, preferably at least 30, and more preferably at least 50, contiguous bases that correspond to, or are complementary to, nucleic acid sequences from the appropriate encoding gene (SEQ ID NO;1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23 and SEQ ID NO:25), are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and east artificial chromosome DNA (Lagerstrom, M. et al. (1991) PCR Methods Applic., 1, 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

Gene silencing approaches may also be undertaken to down-regulate endogenous expression of a gene encoding a polypeptide of the invention. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The vectors of the present invention comprise nucleic acid molecules of the invention and may be cloning or expression vectors. The host cells of the invention, which may be transformed, transfected or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al. (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al., (supra). Particularly suitable methods include calcium phosphate transfection, DEAE-dextran mediated transfection, transfection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSportl™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammnalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk⁻ or aprt^{±} cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al. (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immnobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Inmmunology 1(2):Chapter 5 (1991).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

A preferred method for identifying an agonist or antagonist compound of a polypeptide of the present invention comprises:

(a) contacting a cell expressing on the surface thereof the polypeptide according to the first aspect of the invention, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by measuring the level of a signal generated from the interaction of the compound with the polypeptide.

A further preferred method for identifying an agonist or antagonist of a polypeptide of the invention comprises:

(a) contacting a cell expressing on the surface thereof the polypeptide, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying an agonist or antagonist of a polypeptide of the present invention comprises:

determining the inhibition of binding of a ligand to cells which have a polypeptide of the invention on the surface thereof, or to cell membranes containing such a polypeptide, in the presence of a candidate compound under conditions to permit binding to the polypeptide, and determining the amount of ligand bound to the polypeptide. A compound capable of causing reduction of binding of a ligand is considered to be an agonist or antagonist. Preferably the ligand is labelled.

More particularly, a method of screening for a polypeptide antagonist or agonist compound comprises the steps of:

(a) incubating a labelled ligand with a whole cell expressing a polypeptide according to the invention on the cell surface, or a cell membrane containing a polypeptide of the invention,

(b) measuring the amount of labelled ligand bound to the whole cell or the cell membrane;

(c) adding a candidate compound to a mixture of labelled ligand and the whole cell or the cell membrane of step (a) and allowing the mixture to attain equilibrium;

(d) measuring the amount of labelled ligand bound to the whole cell or the cell membrane after step (c); and

(e) comparing the difference in the labelled ligand bound in step (b) and (d), such that the compound which causes the reduction in binding in step (d) is considered to be an agonist or antagonist.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured using assays such as ProCheck™ Universal Protease Assay (Serologicals Corporation), EnzChek Protease Assay (Molecular Probes) or Protease Activity Detection Kit (PanVera).

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (i.e. to prevent infection) or therapeutic (i.e. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO00/29428.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al, Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:
a)contacting a sample of tissue from the patient with a nucleic acid probe under stringent conditions that allow the formation of a hybrid complex between a nucleic acid molecule of the invention and the probe;
b)contacting a control sample with said probe under the same conditions used in step a);
c)and detecting the presence of hybrid complexes in said samples;
   wherein detection of levels of the hybrid complex in the patient sample that differ from levels of the hybrid complex in the control sample is indicative of disease.

A further aspect of the invention comprises a diagnostic method comprising the steps of:
a)obtaining a tissue sample from a patient being tested for disease;
b)isolating a nucleic acid molecule according to the invention from said tissue sample; and
c)diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformnational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application W095/l 1995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application W095/25116 (Baldeschweiler et al.). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;

(b) a polypeptide of the present invention; or

(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease in which members of the trypsin family of proteins are implicated. Such diseases may include cell proliferative disorders and/or differentiative disorders, such as carcinoma, sarcoma, solid tumours including prostate, breast, lung, testis, ovary, head and neck, brain or bone tumour, metastatic disorders or hematopoietic neoplastic disorders; disorders of bone metabolism, such as osteoporosis, osteodystrophy, osteomalacia, rickets, osteitis fibrosa cystica, renal osteodystrophy, osteosclerosis, osteopenia, fibrogenesis-imperfecta ossium, secondary hyperparathyrodism, hypoparathyroidism, hyperparathyroidism; periodontal disease, immune/autoimmune disorders such as, arthritis, multiple sclerosis, encephalomyelitis, myasthenia gravis, systemic lupus erythematosis, autoimmune thyroiditis, dermatitis, psoriasis, Sjogren's Syndrome, Crohn's disease, ulcer, iritis, conjunctivitis, ulcerative colitis, asthma, scieroderma, autoimmune uveitis, allergic encephalomyelitis, hearing loss, aplastic anemia, anemia, idiopathic thrombocytopenia, polychondritis, Graves' disease, graft-versus-host disease, allergy; hematopoietic disorders; cardiovascular disorders such as hypertension, atherosclerosis, congestive heart failure, coronary artery disease, arrhythmias, cardiomyopathies, artherosclerosis, hypertensive vascular disease, Raynaud disease, aneurysms, varicose veins, thrombophlebitis, phlebothrombosis, tumors, hemangioma, lymphangioma, glomus tumor (glomangioma), Kaposi sarcoma, angiosarcoma, hemangiopericytoma; blood clotting disorders, such as hemorrhagic diatheses, nonthrombocytopenic purpuras, thrombocytopenia, idiopathic thrombocytopenic purpura, HIV-associated thrombocytopenia, thrombotic microangiopathies, hemorrhagic diatheses, lymphomas, Hodgkin disease; liver disorders such as, cirrhosis, hepatocellular necrosis, liver fibrosis, Al-antitrypsin deficiency, hemochromatosis copper storage diseases and liver damage (especially drug or alcohol induced); bacterial, fungal or viral infection; pain or metabolic disorders, such as obesity, anorexia nervosa, cachexia, lipid disorders, and diabetes; AIDS and other pathological conditions.

Preferably, the diseases are those in which members of the trypsin family are implicated.

Various aspects and embodiments of the present invention will now be described in more detail by way of example, with particular reference to the INTP039 polypeptides.

It will be appreciated that modification of detail may be made without departing from the scope of the invention.

### BRIEF DESCRIPTION OF THE FIGURES

FIG. 1: BLASTP output for INTP039 polypeptide sequence (SEQ ID NO;22) against the NCBI non-redundant database.

FIG. 2: Alignment between INTP039 polypeptide sequence (SEQ ID NO:22) and the BLASTP hit, NP_{—}114154.1 (Homo sapiens).

FIG. 3: SigP cleavage site prediction for INTP039.

FIG. 4: INTP039 domain organisation

FIG. 5: INTP039 polypeptide multiple sequence alignment

FIG. 6: Predicted model of INTP039 domains 1 and 2

### EXAMPLES

### Example 1

### INTP039 BLAST Results

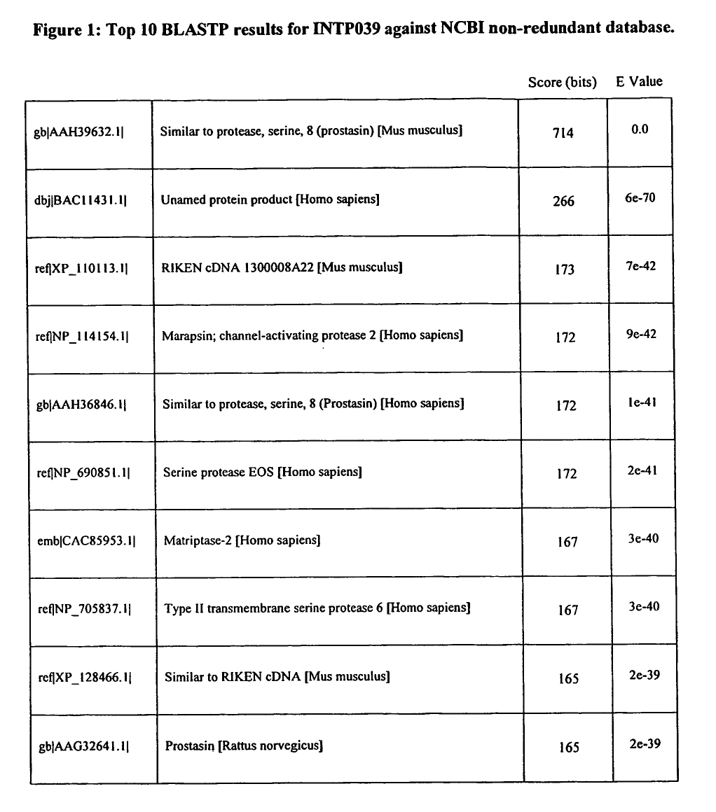

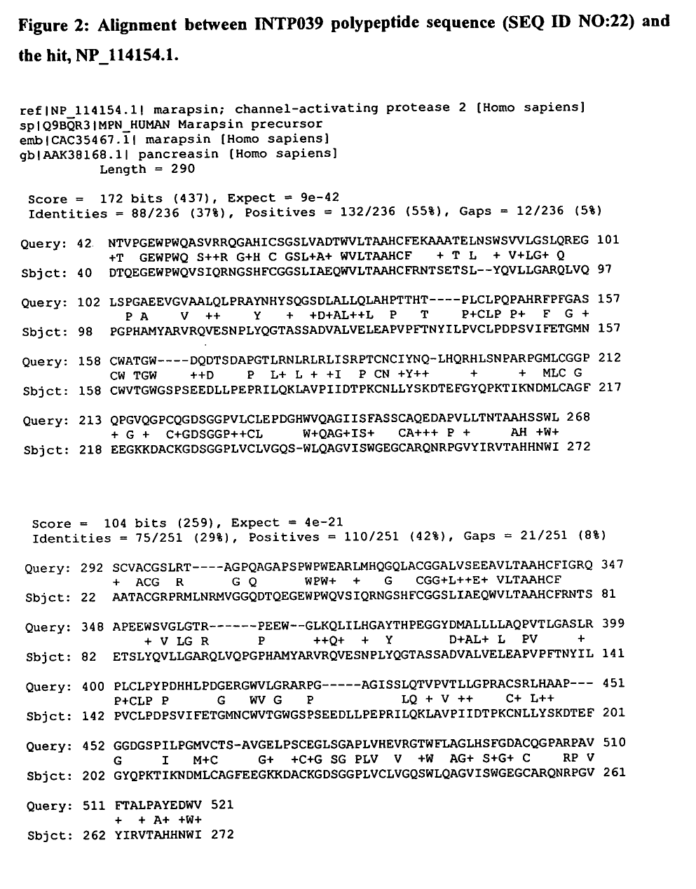

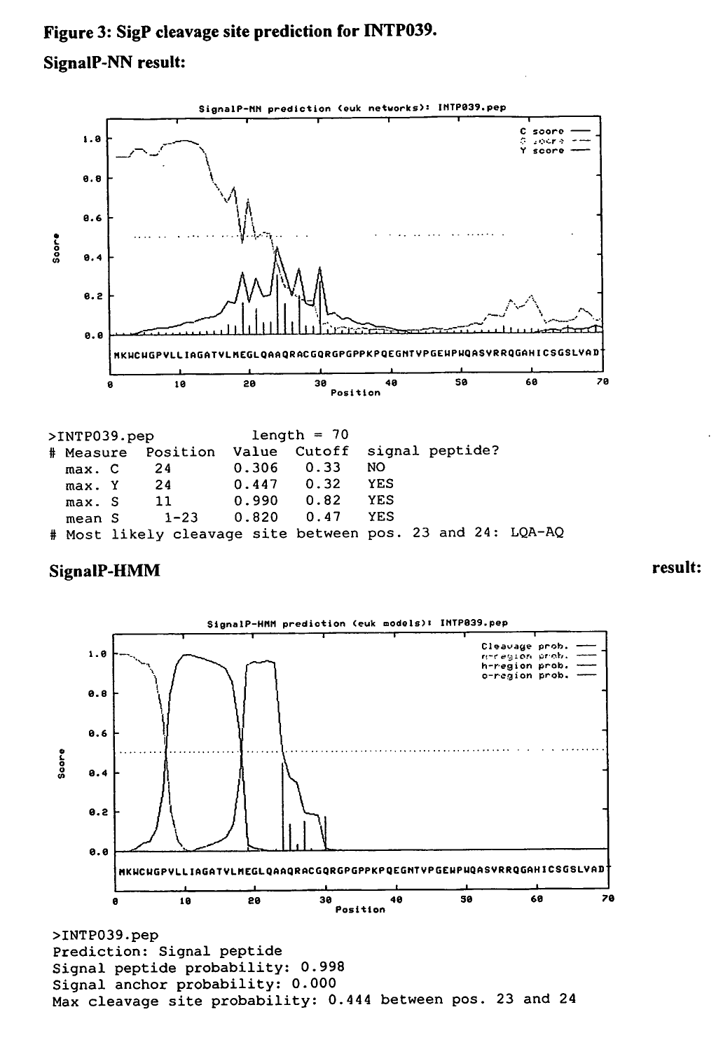

The INTP039 polypeptide sequence (SEQ ID NO:22) was used as a protein BLAST query sequence against the NCBI non-redundant sequence database. FIG. 1 shows the top ten results for the BLASTP query, and FIG. 2 shows the alignment against NP_{—}114154.1 (Homo sapiens).

### Example 2

### INTP039 Signal Sequence

FIG. 3 show that INTP039 is predicted to possess a signal peptide at the start of the protein. As the SigP data in FIG. 3 clearly shows, the signal peptide cleavage site is thought to be between residues 23 and 24 of the INTP039 polypeptide sequence (Nielsen, H. et al. 1997, Protein Engineering, 10, 1-6; Nielsen, H., and Krogh, A.: Prediction of signal peptides and signal anchors by a hidden Markov model. In Proceedings of the Sixth International Conference on Intelligent Systems for Molecular Biology (ISMB 6), AAAI Press, Menlo Park, Calif., pp. 122-130 (1998)).

### Example 3

### INTP039 Structure

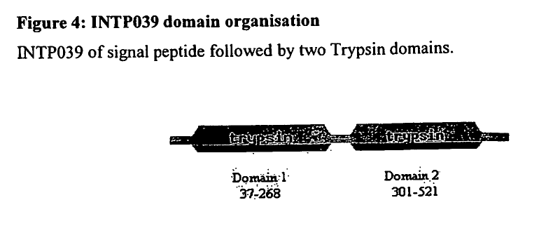

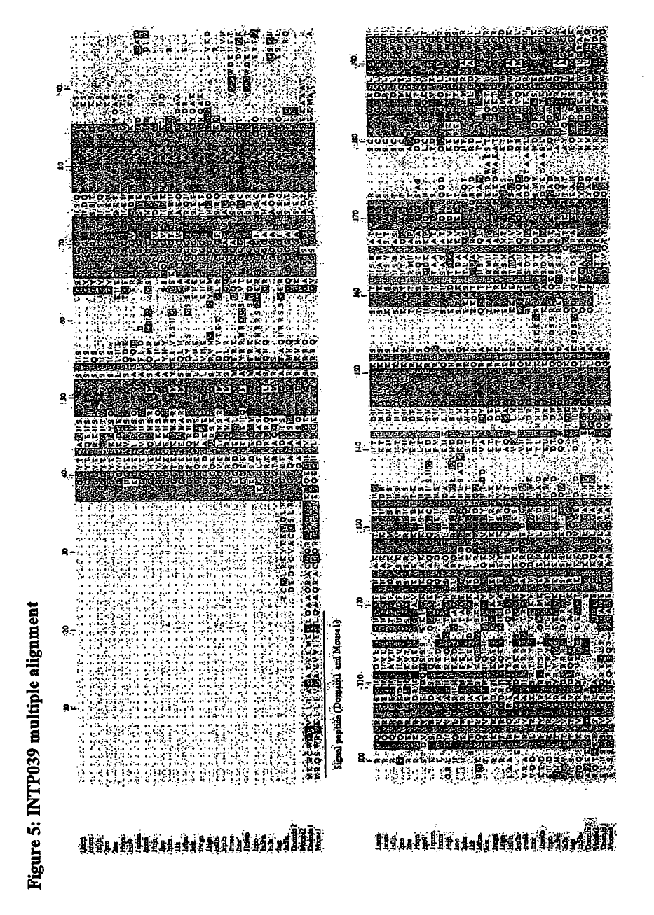

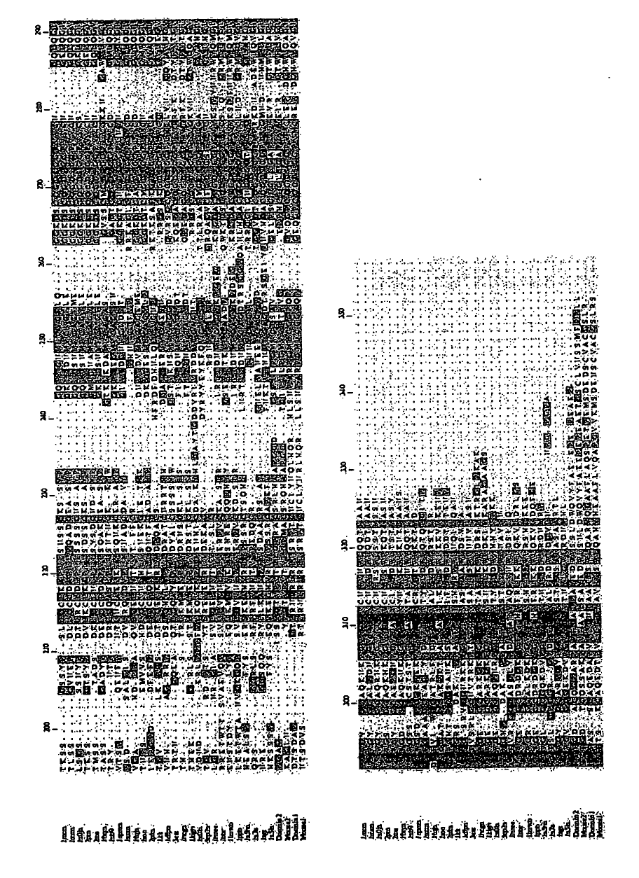

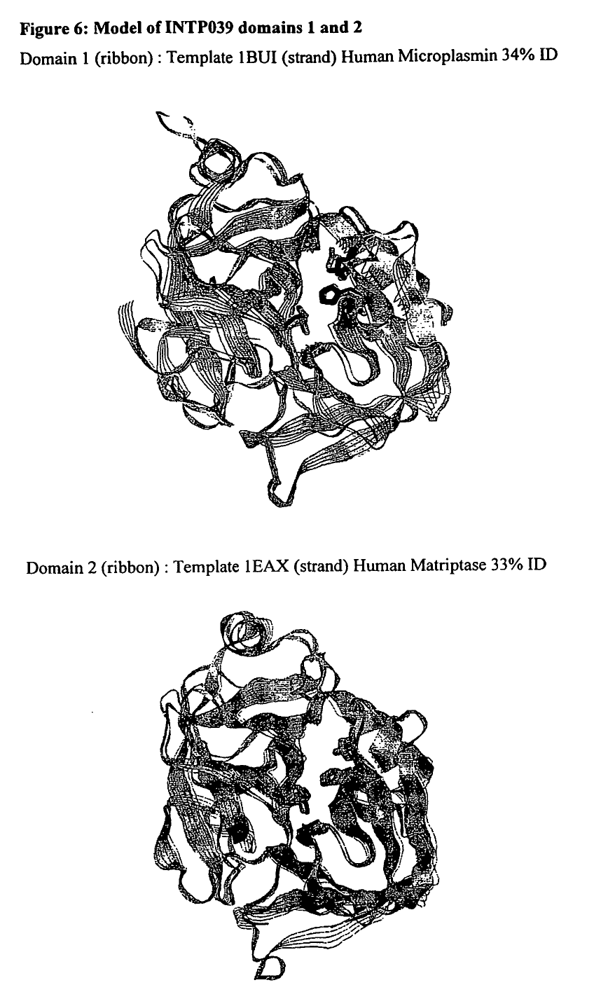

FIG. 4 shows that it is predicted that the INTP039 polypeptide contains two trypsin domains. FIG. 5 shows the predicted structure of the two trypsin domains that are found within INTP039. FIG. 6 shows INTP039 aligned against other proteins that contain such domains.

### Example 4

### EST Data

Table 2 describes expressed sequence tags (ESTs) which have homology to the INTP039 transcript. Based on the cDNA libraries from which the EST's originate, INTP039 appears to be expressed in the tissues listed in Table 1.

TABLE 2
ESTs identified as having homology with INTP039

| Accession number | Source | Organism |
|---|---|---|
| BE792289 | Lung | Homo sapiens |
| Z42936 | Brain | Homo sapiens |
| R18052 | Brain | Homo sapiens |
| BE264142 | Lung | Homo sapiens |
| BQ437135 | Eye | Homo sapiens |

List of INTP039 Specific Sequences:

SEQ ID NO: 1 (INTP039 nucleotide sequence exon 1)

| | |
|---|---|
| 1 | ATGAAGTGGT GCTGGGGCCC AGTGCTGCTC ATCGCGGGTG |
| | CCACAGTCCT |
| 51 | CATGGAGG |

SEQ ID NO: 2 (INTP039 polypeptide sequence exon 1)

| | |
|---|---|
| 1 | MKWCWGPVLL IAGATVLMEG |

SEQ ID NO: 3 (INTP039 nucleotide sequence exon 2)

| | |
|---|---|
| 1 | GTCTTCAAGC CGCTCAGCGT G |

SEQ ID NO: 4 (INTP039 polypeptide sequence exon 2)

| | |
|---|---|
| 1 | LQAAQRA |

SEQ ID NO: 5 (INTP039 nucleotide sequence exon 3)

| | |
|---|---|
| 1 | CCTGTGGACA GCGTGGCCCC GGCCCCCCCA AGCCTCAGGA |
| | GGGCAACACA |
| 51 | GTCCCTGGCG AGTGGCCCTG GCAGGCCAGT GTGAGGAGGC |
| | AAGGAGCCCA |
| 101 | CATCTGCAGC GGCTCCCTGG TGGCAGACAC CTGGGTCCTC |
| | ACTCCTGCCC |
| 151 | ACTGCTTTGA AAA |

SEQ ID NO: 6 (INTP039 polypeptide sequence exon 3)

| | |
|---|---|
| 1 | CGQRGPGPPK PQEGNTVPGE WPWQASVRRQ GAHIGSGSLV |
| | ADTWVLTAAH |
| 51 | CFEK |

SEQ ID NO: 7 (INTP039 nucleotide sequence exon 4)

| | |
|---|---|
| 1 | GGCAGCAGCA ACAGAACTGA ATTCCTGGTC AGTGGTCGTG |
| | GGTTCTCTGC |
| 51 | AGCGTGAGGG ACTCAGCCCT GGGGCCGAAG AGGTGGGGGT |
| | GGCTGCCCTG |
| 101 | CAGTTGCCCA GGGCCTATAA CCACTACAGC CAGGGCTCAG |
| | ACCTGGCCCT |
| 151 | GCTGCAGCTC GCCCACCCCA CGACCCACAC AGCCCTCTGC |
| | CTGCCCCAGC |
| 201 | CCGCCCATCG CTTCCCCTTT GGAGCCTCCT GCTGGGCCAC |
| | TGGCTGGGAT |
| 251 | CAGGACACCA GTGATG |

SEQ ID NO: 8 (INTP039 polypeptide sequence exon 4)

| | |
|---|---|
| 1 | AAATELNSWS VVLGSLQREG LSPGAEEVGV AALQLPRAYN |
| | HYSQGSDLAL |
| 51 | LQLAHPTTHT PLCLPQPAHR FPFGASCWAT GWDQDTSDA |

SEQ ID NO: 9 (INTP039 oucleotide sequence exon 5)

| | |
|---|---|
| 1 | CTCCTGGGAC CCTACGCAAT CTGCGCCTGC GTCTCATCAG |
| | TCGCCCCACA |
| 51 | TGTAACTGTA TCTACAACCA GCTGCACCAG CGACACCTGT |
| | CCAACCCGGC |
| 101 | CCGGCCTGGG ATGCTATGTG GGGGCCCCCA GCCTGGGGTG |
| | CAGGGCCCCT |
| 151 | GTCAG |

SEQ ID NO: 10 (INTP039 polypeptide sequence exon
5)

| | |
|---|---|
| 1 | PGTLRNLRLR LISRPTCNCI YNQLHQRHLS NPARPGMLCG |
| | GPQPGVQGPC |
| 51 | Q |

SEQ ID NO: 11 (INTP039 nucleotide sequence exon 6)

| | |
|---|---|
| 1 | GGAGATTCCG GGGGCCCTGT GCTGTGCCTC GAGCCTGACG |
| | GACACTGGGT |
| 51 | TCAGGCTGGC ATCATCAGCT TTGCATCAAG CTGTGCCCAG |
| | GAGGACGCTC |
| 101 | CTGTGCTGCT GACCAACACA GCTGCTCACA GTTCCTGGCT |
| | GCAGGCTCGA |
| 151 | GTTCAGGGGG CAGCTTTCCT GGCCCAGAGC CCAGAGACCC |
| | CGGAGATGAG |
| 201 | TGATGAGGAC AGCTGTGTAG |

SEQ ID NO: 12 (INTP039 polypeptide sequence exon
6)

| | |
|---|---|
| 1 | GDSGGPVLCL EPDGHWVQAG IISFASSCAQ EDAPVLLTNT |
| | AAHSSWLQAR |
| 51 | VQGAAFLAQS PETPEMSDED SCVA |

SEQ ID NO: 13 (INTP039 nucleotide sequence exon 7)

| | |
|---|---|
| 1 | CCTGTGGATC CTTGAGGACA GCAGGTCCCC AGGCAGGAGC |
| | ACCGTCGCCA |
| 51 | TGGCCCTGGG AGGCCAGGCT GATGCACCAG GGACAGCTGG |
| | CGTGTGGCGG |
| 101 | AGCCCTGGTG TCAGAGGAGG CGGTGCTAAC TGCTGCCCAC |
| | TGCTTCATTG |
| 151 | G |

SEQ ID NO: 14 (INTP039 polypeptide sequence exon
7)

| | |
|---|---|
| 1 | CGSLRTAGPQ AGAPSPWPWE ARLMHQGQLA CGGALVSEEA |
| | VLTAAHCFIG |

SEQ ID NO: 15 (INTP039 nucleotide sequence exon 8)

| | |
|---|---|
| 1 | GCGCCAGGCC CCAGAGGAAT GGAGCGTAGG GCTGGGGACC |
| | AGACCGGAGG |
| 51 | AGTGGGGCCT GAAGCAGGTC ATCCTGCATG GAGCCTACAC |
| | CCACCCTGAG |
| 101 | GGGGGCTACG ACATGGCCCT CCTGCTGCTG GCCCAGCCTG |
| | TGACACTGGG |
| 151 | AGCCAGCCTG CGGCCCCTCT GCCTGCCCTA TCCTGACCAC |
| | CACCTGCCTG |
| 201 | ATGGGGAGCG TGGCTGGGTT CTGGGACGGG CCCGCCCAGG |
| | AGGAG |

SEQ ID NO: 16 (INTP039 polypeptide sequence exon
8)

| | |
|---|---|
| 1 | RQAPEEWSVG LGTRPEEWGL KQLILHGAYT HPEGGYDMAL |
| | LLLAQPVTLG |
| 51 | ASLRPLCLPY PDHHLPDGER GWVLGRARPG AG |

SEQ ID NO: 17 (INTP039 nucleotide sequence exon 9)

| | |
|---|---|
| 1 | GCATCAGCTC CCTCCAGACA GTGCCCGTGA CCCTCCTGGG |
| | GCCTAGGGCC |
| 51 | TGCAGCCGGC TGCATGCAGC TCCTGGGGGT GATGGCAGCC |
| | CTATTCTGCC |
| 101 | GGGGATGGTG TGTACCAGTG CTGTGGGTGA GCTGCCCAGC |
| | TGTGAG |

SEQ ID NO: 18 (INTP039 polypeptide sequence exon
9)

| | |
|---|---|
| 1 | ISSLQTVPVT LLGPRACSRL HAAPGGDGSP ILPGMVCTSA |
| | VGELPSCE |

SEQ ID NO: 19 (INTP039 nucleotide sequence exon
10)

| | |
|---|---|
| 1 | GGCCTGTCTG GGGCACCACT GGTGCATGAG GTGAGGGGCA |
| | CATGGTTCCT |
| 51 | GGCCGGGCTG CACAGCTTCG GAGATGCTTG CCAAGGCCCC |
| | GCCAGGCCGG |
| 101 | CGGTCTTCAC CGCGCTCCCT GCCTATGAGG ACTGGGTCAG |
| | CAGTTTGGAC |
| 151 | TGGCAGGTCT ACTTCGCCGA GGAACCAGAG CCCGAGGCTG |
| | AGCCTGGAAG |
| 201 | CTGCGTGGCC AACATAAGTA TGTGGCCCCG GGGCCTCCTG |
| | CCAAACCCTG |
| 251 | CCTCTCCAGG ACCCTTCTCT CTGCAG |

SEQ ID NO: 20 (INTP039 polypeptide sequence exon
10)

| | |
|---|---|
| 1 | GLSGAPLVHE VRGTWFLAGL HSFGDACQGP ARPAVFTALP |
| | AYEDWVSSLD |
| 51 | WQVYFAEEPE PEAEPGSCLA NISMWPRGLL PNPASPGPFS |
| | LQ |

SEQ ID NO: 21 (INTP039 nucleotide sequence)

| | |
|---|---|
| 1 | ATGAAGTGGT GCTGGGGCCC AGTGCTGCTC ATCGCGGGTG |
| | CCACAGTCCT |
| 51 | CATGGAGGGT CTTCAAGCCG CTCAGGGTGC CTGTGGACAG |
| | CGTGGCCCCG |
| 101 | GCCCCCCCAA GCCTCAGGAG GGCAACACAG TCGCTGGCGA |
| | GTGGCCCTGG |
| 151 | CAGGCCAGTG TGAGGAGGCA AGGAGCCCAC ATCTGCAGCG |
| | GCTCCCTGGT |
| 201 | GGCAGACACC TGGGTCCTCA CTGCTGCCCA CTGCTTTGAA |
| | AAGGCAGCAG |
| 251 | CAACAGAACT GAATTCCTGG TCAGTGGTCC TGGGTTCTCT |
| | GCAGCGTGAG |
| 301 | GGACTCAGCC CTGGGGCCGA AGAGGTGGGG GTGGCTGCCC |
| | TGCAGTTGCC |
| 351 | CAGGGCCTAT AACCACTACA GCCAGGGCTC AGACCTGGCC |
| | CTGCTGCAGC |
| 401 | TCGCCCACCC CACGACCCAC ACACCCCTCT GCCTGCCCCA |
| | GCCCGCCCAT |
| 451 | CGCTTCCCCT TTGGAGCCTC CTGCTGGGCC ACTGGCTGGG |
| | ATCAGGACAC |
| 501 | CAGTGATGCT CCTGGGACCC TACGCAATCT GCGCCTGCGT |
| | CTCATCAGTC |
| 551 | GCCCCACATG TAACTGTATC TACAACCAGC TGCACCAGCG |
| | ACACCTGTCC |
| 601 | AACCCGGCCC GGCCTGGGAT GCTATGTGGG GGCCCCCAGC |
| | CTGGGGTGCA |
| 651 | GGGCCCCTGT CAGGGAGATT CCGGGGGCCC TGTGCTGTGC |
| | CTCGAGCCTG |
| 701 | ACGGACACTG GGTTCAGGCT GGCATCATCA GCTTTGCATC |
| | AAGCTGTGCC |
| 751 | CAGGAGGACG CTCCTGTGCT GCTGACCAAC ACAGCTGCTC |
| | ACAGTTCCTG |
| 801 | GCTGCAGGCT CGAGTTCAGG GGGCAGCTTT CCTGGCCCAG |
| | AGCCCAGAGA |
| 851 | CCCCGGAGAT GAGTGATGAG GACAGCTGTG TAGCCTGTGG |
| | ATCCTTGAGG |
| 901 | ACAGCAGGTC CCCAGGCAGG AGCACCCTCC CCATGGCCCT |
| | GGGAGGCCAG |
| 951 | GCTGATGCAC CAGGGACAGC TGGCCTGTGG CGGAGCCCTG |
| | GTGTCAGAGG |
| 1001 | AGGCGGTGCT AACTGCTGCC CACTGCTTCA TTGGGCGCCA |
| | GGCCCCAGAG |
| 1051 | GAATGGAGCG TAGGGCTGGG GACCAGACCG GAGGAGTGGG |
| | GCCTGAAGCA |
| 1101 | GCTCATCCTG CATGGAGCCT ACACCCACCC TGAGGGGGGC |
| | TACGACATGG |
| 1151 | CCCTCCTGCT GCTGGCCCAG CCTGTGACAC TGGGAGCCAG |
| | CCTGCGGCCC |
| 1201 | CTCTGCCTGC CCTATCCTGA CCACCACCTG CCTGATGGGG |
| | AGCGTGGCTG |
| 1251 | GGTTCTGGGA CGGGCCCGCC CAGGAGCAGG CATCAGCTCC |
| | CTCCAGACAG |
| 1301 | TGCCCGTGAC CCTCCTGGGG CCTAGGGCCT GCAGCCGGCT |
| | GCATGCAGCT |
| 1351 | CCTGGGGGTG ATGGCAGCCC TATTCTGCCG GGGATGGTGT |
| | GTACCAGTGC |
| 1401 | TGTGGGTGAG CTGCCCAGCT GTGAGGGCCT GTCTGGGGCA |
| | CCACTGGTGC |
| 1451 | ATGAGGTGAG GGGCACATGG TTCCTGGCCG GGCTGCACAG |
| | CTTCGGAGAT |
| 1501 | GCTTGGCAAG GCCCCGCCAG GCCGGCGGTC TTCACCGCGC |
| | TCCCTGCCTA |
| 1551 | TGAGGACTGG GTCAGCAGTT TGGACTGGCA GGTCTACTTC |
| | GCCGAGGAAC |
| 1601 | CAGAGCCGGA GGCTGAGCCT GGAAGCTGCC TGGCCAACAT |
| | AAGTATGTGG |
| 1651 | CCCCGGGGCC TCCTGCCAAA CCCTGCCTCT CCAGGACCCT |
| | TCTCTCTCCA |
| 1701 | G |

SEQ ID NO: 22 (INTP039 polypeptide sequence)

| | |
|---|---|
| 1 | MKWCWGPVLL IAGATVLMEG LQAAQRACGQ RGPGPPKPQE |
| | GNTVPGEWPW |
| 51 | QASVRRQGAH ICSGSLVADT WVLTAAHCFE KAAATELNSW |
| | SVVLGSLQRE |
| 101 | GLSPGAEEVG VAALQLPRAY NHYSQGSDLA LLQLAHPTTH |
| | TPLCLPQPAH |
| 151 | RFPFGASCWA TGWDQDTSDA PGTLRNLRLR LISRPTCNCI |
| | YNQLHQRHLS |
| 201 | NPARPGMLCG GPQPGVQGPC QGDSGGPVLC LEPDGHWVQA |
| | GIISFASSCA |
| 251 | QEDAPVLLTN TAAHSSWLQA RVQGAAFLAQ SPETPEMSDE |
| | DSCVACGSLR |
| 301 | TAGPQAGAPS PWPWEARLMH QGQLACGGAL VSEEAVLTAA |
| | HCFIGRQAPE |
| 351 | EWSVGLGTRP EEWGLKQLIL HGAYTHPEGG YDMALLLLAQ |
| | PVTLGASLRP |
| 401 | LCLPYPDHHL PDGERGWVLG RARPGAGISS LQTVPVTLLG |
| | PRACSRLHAA |
| 451 | PGGDGSPILP GMVCTSAVGE LPSCEGLSGA PLVHEVRGTW |
| | FLAGLHSFGD |
| 501 | ACQGPARPAV FTALPAYEDW VSSLDWQVYF AEEPEPEAEP |
| | GSCLANISMW |
| 551 | PRGLLPNPAS PGPFSLQ |

SEQ ID NO: 23 (INTP039 exon 2 mature nucleotide
sequence)

| | |
|---|---|
| 1 | GCTCAGCGTG |

SEQ ID NO: 24 (INTP039 exon 2 mature polypeptide
sequence)

| | |
|---|---|
| 1 | AQRA |

SEQ ID NO: 25 (INTP039 mature nucleotide sequence)

| | |
|---|---|
| 1 | GCTCAGCGTG CCTGTGGACA GCGTGGCCCC GGCCCCCCCA |
| | AGCCTCAGGA |
| 51 | GGGCAACACA GTCCCTGGCG AGTGGCCCTG GCAGGCCAGT |
| | GTGAGGAGGC |
| 101 | AAGGAGCCCA CATCTGCAGC GGCTCCCTGG TGGCAGACAC |
| | CTGGGTCCTC |
| 151 | ACTGCTGCCC ACTGCTTTGA AAAGGCAGCA GCAACAGAAC |
| | TGAATTCCTG |
| 201 | GTCAGTGGTC CTGGGTTCTC TGCAGCGTGA GGGACTCAGC |
| | CCTGGGGCCG |
| 251 | AAGAGGTGGG GGTGGCTGCC CTGCAGTTGC CCAGGGCCTA |
| | TAACCACTAC |
| 301 | AGCCAGGGCT CAGACCTGGC CCTGCTGCAG CTCGCCCACC |
| | CCACGACCCA |
| 351 | CACACCCCTC TGCCTGCCCC AGCCCGCCCA TCGCTTCCCC |
| | TTTGGAGCCT |
| 401 | CCTGCTGGGC CACTGGCTGG GATCAGGACA CCAGTGATGC |
| | TCCTGGGACC |
| 451 | CTACGCAATC TGCGGCTGCG TCTCATCAGT CGCGCCACAT |
| | GTAACTGTAT |
| 501 | CTACAACCAG CTGCACCAGC GACACCTGTC CAACCCGGCC |
| | CGGCCTGGGA |
| 551 | TGCTATGTGG GGGCCCCCAG CCTGGGGTGC AGGGCCCCTG |
| | TCAGGGAGAT |
| 601 | TCCGGGGGCC CTGTGCTGTG CCTCGAGCCT GACGGACACT |
| | GGGTTCAGGC |
| 651 | TGGCATCATC AGCTTTGCAT CAAGCTGTGC CCAGGAGGAC |
| | GCTCCTGTGC |
| 701 | TGCTGACCAA CACAGCTGCT CACAGTTCCT GGCTGCAGGC |
| | TCGAGTTCAG |
| 751 | GGGGCAGCTT TCCTGGCCCA GAGCCCAGAG ACCCCGGAGA |
| | TGAGTGATGA |
| 801 | GGACAGCTGT GTAGCCTGTG GATCCTTGAG GACAGCAGGT |
| | CCCCAGGCAG |
| 851 | GAGCACCCTC GCCATGGCCC TGGGAGGCCA GGCTGATGCA |
| | CCAGGGACAG |
| 901 | CTGGCCTGTG GCGGAGCCCT GGTGTCAGAG GAGGCGGTGC |
| | TAACTGCTGC |
| 951 | CCACTGCTTC ATTGGGCGCC AGGCCCCAGA GGAATGGAGC |
| | GTAGGGCTGG |
| 1001 | GGACCAGACC GGAGGAGTGG GGCCTGAAGC AGCTCATCCT |
| | GCATGGAGCC |
| 1051 | TACACCCAGG CTGAGGGGGG CTACGACATG GCGCTCCTGC |
| | TGCTGGCCCA |
| 1101 | GCCTGTGACA CTGGGAGCCA GCCTGCGGCC CCTCTGCCTG |
| | CCCTATCCTG |
| 1151 | ACCACCACGT GCCTGATGGG GAGCGTGGCT GGGTTCTGGG |
| | ACGGGCCCGC |
| 1201 | CCAGGAGCAG GGATCAGCTC CCTCCAGACA GTGCCCGTGA |
| | CCCTCCTGGG |
| 1251 | GCCTAGGGCC TGCAGCCGGC TGCATGCAGC TCCTGGGGGT |
| | GATGGCAGCC |
| 1301 | CTATTCTGCC GGGGATGGTG TGTACCAGTG CTGTGGGTGA |
| | GCTGCCCAGC |
| 1351 | TGTGAGGGCC TGTCTGGGGC ACCACTGGTG CATGAGGTGA |
| | GGGGCACATG |
| 1401 | GTTCCTGGCC GGGCTGCACA GCTTCGGAGA TGCTTGCCAA |
| | GGCCCCGCCA |
| 1451 | GGCCGGCGGT CTTCACCGCG CTCCCTGCCT ATGAGGACTG |
| | GGTCAGCAGT |
| 1501 | TTGGACTGGC AGGTCTACTT CGCCGAGGAA CCAGAGCCCG |
| | AGGCTGAGCC |
| 1551 | TGGAAGCTGC CTGGCCAACA TAAGTATGTG GCCCCGGGGC |
| | CTCCTGCCAA |
| 1601 | ACCCTGCCTC TCCAGGACCC TTCTCTCTCC AG |

SEQ ID NO: 26 (INTP039 mature polypeptide
sequence)

| | |
|---|---|
| 1 | AQRACGQRGP GPPKPQEGNT VPGEWPWQAS VRRQGAHICS |
| | GSLVADTWVL |
| 51 | TAAHCFEKAA ATELNSWSVV LGSLQREGLS PGAEEVGVAA |
| | LQLPRAYNHY |
| 101 | SQGSDLALLQ LAHPTTHTPL CLPQPAHRFP FGASCWATGW |
| | DQDTSDAPGT |
| 151 | LRNLRLRLIS RPTCNCIYNQ LHQRHLSNPA RPGMLCGGPQ |
| | PGVQGPCQGD |
| 201 | SGGPVLCLEP DGHWVQAGII SFASSCAQED APVLLTNTAA |
| | HSSWLQARVQ |
| 251 | GAAFLAQSPE TPEMSDEDSC VACGSLRTAG PQAGAPSPWP |
| | WEARLHHQGQ |
| 301 | LACGGALVSE EAVLTAAHCF IGRQAPEEWS VGLGTRPEEW |
| | GLKQLILHGA |
| 351 | YTHPEGGYDM ALLLLAQPVT LGASLRPLCL PYPDHHLPDG |
| | ERGWVLGRAR |
| 401 | PGAGISSLQT VPVTLLGPRA CSRLHAAPGG DGSPTLPGMV |
| | CTSAVGELPS |
| 451 | CEGLSGAPLV HEVRGTWFLA GLHSFGDACQ GPARPAVFTA |
| | LPAYEDWVSS |
| 501 | LDWQVYFAEE PEPEAEPGSC LANISMWPRG LLPNPASPGP |
| | FSLQ |

We claim:

1-47. (canceled)

48. A composition of matter comprising:
a) an isolated polypeptide selected from the group consisting of:
1) an amino acid sequence comprising that selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ 1 DNO:22, SEQ ID NO:24, and SEQ ID NO:26;
2) a fragment of said amino acid sequence which functions as a serine protease which is a member of the trypsin family of proteins, or having an antigenic determinant in common with a polypeptide according to 1);
3) a functional equivalent of 1) or 2);
4) an amino acid sequence consisting of that selected from the group consisting of SEQ ID NO:2, SEQ ID NO:4, SEQ ID NO:6, SEQ ID NO:8, SEQ ID NO: 10, SEQ ID NO;12, SEQ ID NO:14, SEQ ID NO:16, SEQ ID NO:18, SEQ ID NO:20, SEQ 1 DNO:22, SEQ ID NO:24, and SEQ ID NO:26;
5) a fragment of 4), wherein the fragment functions as a serine protease which is a member of the trypsin family of proteins, or having an antigenic determinant in common with a polypeptide according to 4);
6) the functional equivalent of 3), wherein the functional equivalent is homologous to an amino acid sequence selected from the group consisting of SEQ ID NO:2, SEQ ID NO:4, SEQ ID NO:6, SEQ ID NO:8, SEQ ID NO;10, SEQ ID NO;12, SEQ ID NO;14, SEQ ID NO:16, SEQ ID NO:18, SEQ ID NO:20, SEQ 1 DNO:22, SEQ ID NO:24, and SEQ ID NO:26, and is a member of the trypsin family of proteins;
7) the fragment of 2), wherein the fragment has greater than 80% sequence identity with an amino acid sequence selected from the group consisting of SEQ ID NO:2, SEQ ID NO:4, SEQ ID NO:6, SEQ ID NO:8, SEQ ID NO;10, SEQ ID NO;12, SEQ ID NO;14, SEQ ID NO:16, SEQ ID NO:18, SEQ ID NO:20, SEQ 1 DNO:22, SEQ ID NO:24, and SEQ ID NO:26, or with an active fragment thereof;
8) the fragment of 2), wherein the fragment has greater than 90% sequence identity with an amino acid sequence selected from the group consisting of SEQ ID NO:2, SEQ ID NO:4, SEQ ID NO:6, SEQ ID NO:8, SEQ ID NO:10, SEQ ID NO;12, SEQ ID NO:14, SEQ ID NO:16, SEQ ID NO:18, SEQ ID NO:20, SEQ ID NO:22, SEQ ID NO:24, and SEQ ID NO:26, or with an active fragment thereof;
9) the functional equivalent of 3), wherein the functional equivalent has greater than 80% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26, or with an active fragment thereof;
10) the functional equivalent of 3), wherein the functional equivalent has greater than 90% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26, or with an active fragment thereof;
11) the functional equivalent of 3), wherein the functional equivalent exhibits significant structural homology with a polypeptide comprising an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ 1 DNO:22, SEQ ID NO:24, and SEQ ID NO:26; and
12) the functional equivalent of 3), wherein the functional equivalent has an antigenic determinant in common with the polypeptide of 1), and wherein the functional equivalent consists of 7 or more amino acid residues from an amino acid sequence selected from the group consisting of SEQ ID NO:2, SEQ ID NO:4, SEQ ID NO:6, SEQ ID NO:8, SEQ ID NO;10, SEQ ID NO;12, SEQ ID NO;14, SEQ ID NO;16, SEQ ID NO;18, SEQ ID NO:20, SEQ I DNO:22, SEQ ID NO:24, and SEQ ID NO:26; or
b) a purified nucleic acid molecule:
1) encoding a polypeptide of any of a1) to a12); or
2) comprising the nucleic acid sequence recited in SEQ ID NO;1, SEQ ID NO:3, SEQ ID NO:5, SEQ ID NO:7, SEQ ID NO:9, SEQ ID NO;11, SEQ ID NO;13, SEQ ID NO;15, SEQ ID NO;17, SEQ ID NO;19, SEQ ID NO:21, SEQ ID NO:23, and SEQ ID NO:25, or is a redundant equivalent or fragment of any of the foregoing; or
3) consisting of the nucleic acid sequence recited in SEQ ID NO;1, SEQ ID NO:3, SEQ ID NO:5, SEQ ID NO:7, SEQ ID NO:9, SEQ ID NO:11, SEQ ID NO;13, SEQ ID NO:15, SEQ ID NO:17, SEQ ID NO:19, SEQ ID NO:21, SEQ ID NO:23, and SEQ ID NO:25, or is a redundant equivalent or fragment of any of the foregoing; or
4) that hybridizes under high stringency conditions with a nucleic acid molecule of any of b1) to b3); or
c) a vector comprising a nucleic acid molecule according to any one of b1) to b4); or
d) a host cell transformed with a vector or a nucleic acid molecule according to any one of b) or c); or
e) a ligand:
1) that binds specifically to the polypeptide of any of a1) to al2); or
2) which is an antibody that binds specifically to the polypeptide of any of a1) to a12); or
f) a compound:
1) that increases the level of expression or activity of a polypeptide according to any of a1) to al2); or
2) that decreases the level of expression or activity of a polypeptide according to any of a1) to al2); or
g) a compound that binds to a polypeptide according to any of a1) to al2) without inducing any of the biological effects of the polypeptide; or
h) a compound that binds to a polypeptide according to any of a1) to al2) without inducing any of the biological effects of the polypeptide, wherein the compound is a natural or modified substrate, ligand, enzyme, receptor or structural or functional mimetic; or
i) a pharmaceutical composition comprising any one of a) to h), and a pharmaceutically acceptable carrier; or
j) a vaccine composition comprising any one of a1) to a12) or b1) to b4); or
k) a kit for diagnosing disease, comprising a first container containing a nucleic acid probe that hybridizes under stringent conditions with a nucleic acid molecule of any one of b1) to b4), a second container containing primers useful for amplifying the nucleic acid molecule, and instructions for using the probe and primers for facilitating the diagnosis of disease; or
l) a kit for diagnosing disease, comprising a first container containing a nucleic acid probe that hybridizes under stringent conditions with a nucleic acid molecule of any one of b1) to b4); a second container containing primers useful for amplifying the nucleic acid molecule; a third container holding an agent for digesting unhybridized RNA; and instructions for using the probe and primers for facilitating the diagnosis of disease; or
m) a kit comprising an array of nucleic acid molecules, at least one of which is a nucleic acid molecule according to any one of b1) to b4); or
n) a kit comprising one or more antibodies that bind to a polypeptide as recited in any one of a1) to a12); and a reagent useful for the detection of a binding reaction between the one or more antibodies and the polypeptide; or
o) a transgenic or knockout non-human animal that has been transformed to express higher, lower, or absent levels of a polypeptide according to any one of a1) to a12).

49. A method of using a composition of matter, comprising obtaining a composition of matter according to claim 48 and using said composition of matter in a method selected from: diagnosing a disease in a patient; treatment of a disease in a patient; monitoring the therapeutic treatment of a disease; identification of a compound that is effective in the treatment and/or diagnosis of a disease; and screening candidate compounds.

50. The method of claim 49, wherein said method of using a composition of matter comprises the method for treatment of a disease, comprising administering to the patient:
a) an isolated polypeptide selected from the group consisting of:
1) an amino acid sequence comprising that selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26;
2) a fragment of said amino acid sequence which functions as a serine protease which is a member of the trypsin family of proteins, or having an antigenic determinant in common with a polypeptide according to 1);
3) a functional equivalent of 1) or 2);
4) an amino acid sequence consisting of that selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26;
5) a fragment of 4), wherein the fragment functions as a serine protease which is a member of the trypsin family of proteins, or having an antigenic determinant in common with a polypeptide according to 4);
6) the functional equivalent of 3), wherein the functional equivalent is homologous to an amino acid sequence selected from the group consisting of SEQ ID NO:2, SEQ ID NO:4, SEQ ID NO:6, SEQ ID NO:8, SEQ ID NO;10, SEQ ID NO;12, SEQ ID NO;14, SEQ ID NO:16, SEQ ID NO:18, SEQ ID NO:20, SEQ ID NO:22, SEQ ID NO:24, and SEQ ID NO:26, and is a member of the trypsin family of proteins;
7) the fragment of 2), wherein the fragment has greater than 80% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26, or with an active fragment thereof;
8) the fragment of 2), wherein the fragment has greater than 90% sequence identity with an amino acid sequence selected from the group consisting of SEQ ID NO:2, SEQ ID NO:4, SEQ ID NO:6, SEQ ID NO:8, SEQ ID NO;10, SEQ ID NO;12, SEQ ID NO;14, SEQ ID NO:16, SEQ ID NO:18, SEQ ID NO:20, SEQ ID NO:22, SEQ ID NO:24, and SEQ ID NO:26, or with an active fragment thereof;
9) the functional equivalent of 3), wherein the functional equivalent has greater than 80% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26, or with an active fragment thereof;
10) the functional equivalent of 3), wherein the functional equivalent has greater than 90% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26, or with an active fragment thereof;
11) the functional equivalent of 3), wherein the functional equivalent exhibits significant structural homology with a polypeptide comprising an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ I DNO:22, SEQ ID NO:24, and SEQ ID NO:26; and
12) the functional equivalent of 3), wherein the functional equivalent has an antigenic determinant in common with the polypeptide of 1), and wherein the functional equivalent consists of 7 or more amino acid residues from an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, and SEQ ID NO:26; or
b) a purified nucleic acid molecule:
1) encoding a polypeptide of any of a1) to a12); or
2) comprising the nucleic acid sequence recited in SEQ ID NO;1, SEQ ID NO:3, SEQ ID NO:5, SEQ ID NO:7, SEQ ID NO:9, SEQ ID NO;11, SEQ ID NO;13, SEQ ID NO;15, SEQ ID NO:17, SEQ ID NO:19, SEQ ID NO:21, SEQ ID NO:23, and SEQ ID NO:25, or is a redundant equivalent or fragment of any of the foregoing; or
3) consisting of the nucleic acid sequence recited in SEQ ID NO;1, SEQ ID NO:3, SEQ ID NO:5, SEQ ID NO:7, SEQ ID NO:9, SEQ ID NO:11, SEQ ID NO;13, SEQ ID NO;15, SEQ ID NO;17, SEQ ID NO;19, SEQ ID NO:21, SEQ ID NO:23, and SEQ ID NO:25, or is a redundant equivalent or fragment of any of the foregoing; or
4) that hybridizes under high stringency conditions with a nucleic acid molecule of any of b1) to b3); or
c) a vector comprising a nucleic acid molecule according to any one of b1) to b4); or
d) a host cell transformed with a vector or a nucleic acid molecule according to any one of b) or c); or
e) a ligand:
1) that binds specifically to the polypeptide of any of a1) to a22); or
2) which is an antibody that binds specifically to the polypeptide of any of a1) to a12); or
f) a compound:
1) that increases the level of expression or activity of a polypeptide according to any of a1) to a12); or
2) that decreases the level of expression or activity of a polypeptide according to any of a1) to a12); or
g) a compound that binds to a polypeptide according to any of a1) to a12) without inducing any of the biological effects of the polypeptide; or
h) a compound that binds to a polypeptide according to any of a1) to a12) without inducing any of the biological effects of the polypeptide, wherein the compound is a natural or modified substrate, ligand, enzyme, receptor or structural or functional mimetic; or
i) a pharmaceutical composition comprising any one of a) to h), and a pharmaceutically acceptable carrier.

51. The method of claim 49, wherein said method of using a composition of matter comprises the method for treatment of a disease, and wherein the disease includes cell proliferative disorders and/or differentiative disorders, such as carcinoma, sarcoma, solid tumours including prostate, breast, lung, testis, ovary, head and neck, brain or bone tumour, metastatic disorders or hematopoietic neoplastic disorders; disorders of bone metabolism, such as osteoporosis, osteodystrophy, osteomalacia, rickets, osteitis fibrosa cystica, renal osteodystrophy, osteosclerosis, osteopenia, fibrogenesis-imperfecta ossium, secondary hyperparathyrodism, hypoparathyroidism, hyperparathyroidism; periodontal disease, immune/autoimmune disorders such as, arthritis, multiple sclerosis, encephalomyelitis, myasthenia gravis, systemic lupus erythematosis, autoimmune thyroiditis, dermatitis, psoriasis, Sjogren's Syndrome, Crohn's disease, ulcer, iritis, conjunctivitis, ulcerative colitis, asthma, scleroderma, autoimmune uveitis, allergic encephalomyelitis, hearing loss, aplastic anemia, anemia, idiopathic thrombocytopenia, polychondritis, Graves' disease, graft-versus-host disease, allergy; hematopoietic disorders; cardiovascular disorders such as hypertension, atherosclerosis, congestive heart failure, coronary artery disease, arrhythmias, cardiomyopathies, artherosclerosis, hypertensive vascular disease, Raynaud disease, aneurysms, varicose veins, thrombophlebitis, phlebothrombosis, tumors, hemangioma, lymphangioma, glomus tumor (glomangioma), Kaposi sarcoma, angiosarcoma, hemangiopericytoma; blood clotting disorders, such as hemorrhagic diatheses, nonthrombocytopenic purpuras, thrombocytopenia, idiopathic thrombocytopenic purpura, HIV-associated thrombocytopenia, thrombotic microangiopathies, hemorrhagic diatheses, lymphomas, Hodgkin disease; liver disorders such as, cirrhosis, hepatocellular necrosis, liver fibrosis, Al-antitrypsin deficiency, hemochromatosis copper storage diseases and liver damage (especially drug or alcohol induced); bacterial, fungal or viral infection; pain or metabolic disorders, such as obesity, anorexia nervosa, cachexia, lipid disorders, and diabetes; AIDS and other pathological conditions.

52. The method of claim 49, wherein said method of using a composition of matter comprises the method for treatment of a disease, and wherein the disease is one for which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

53. The method of claim 49, wherein said method of using a composition of matter comprises the method for treatment of a disease, and wherein the disease is one for which expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

54. The method of claim 47, wherein said method of using a composition of matter comprises the method for diagnosing a disease in a patient, comprising assessing the level of expression of a natural gene encoding a polypeptide of claim 48, or assessing the activity of a polypeptide of claim 48, in tissue from said patient; and comparing said level of expression or activity to a control level, wherein a level that is different to said control level is indicative of disease.

55. The method of claim 54, which is carried out in vitro.

56. The method of claim 54, comprising the steps of:
a) contacting a ligand of claim 48 with a biological sample under conditions suitable for the formation of a ligand-polypeptide complex; and
b) detecting said complex.

57. The method of claim 54, comprising the steps of:
a) contacting a sample of tissue from the patient with a nucleic acid probe under stringent conditions that allow the formation of a hybrid complex between a nucleic acid molecule of claim 48 and the probe;
b) contacting a control sample with said probe under the same conditions used in step a); and
c) detecting the presence of hybrid complexes in said samples; wherein detection of levels of the hybrid complex in the patient sample that differ from levels of the hybrid complex in the control sample is indicative of disease.

58. The method of claim 54, comprising the steps of:
a) contacting a sample of nucleic acid from tissue of the patient with a nucleic acid primer under stringent conditions that allow the formation of a hybrid complex between a nucleic acid molecule of claim 48 and the primer;
b) contacting a control sample with said primer under the same conditions used in step a); and
c) amplifying the sampled nucleic acid; and
d) detecting the level of amplified nucleic acid from both patient and control samples; wherein detection of levels of the amplified nucleic acid in the patient sample that differ significantly from levels of the amplified nucleic acid in the control sample is indicative of disease.

59. The method of claim 54, comprising:
a) obtaining a tissue sample from a patient being tested for disease;
b) isolating a nucleic acid molecule of claim 48 from said tissue sample; and
c) diagnosing the patient for disease by detecting the presence of a mutation which is associated with disease in the nucleic acid molecule as an indication of the disease.

60. The method of claim 59, further comprising amplifying the nucleic acid molecule to form an amplified product and detecting the presence or absence of a mutation in the amplified product.

61. The method of claim 59, wherein the presence or absence of the mutation in the patient is detected by contacting said-nucleic acid molecule with a nucleic acid probe that hybridizes to said nucleic acid molecule under stringent conditions to form a hybrid double-stranded molecule, the hybrid double-stranded molecule having an unhybridized portion of the nucleic acid probe strand at any portion corresponding to a mutation associated with disease; and detecting the presence or absence of an unhybridized portion of the probe strand as an indication of the presence or absence of a disease-associated mutation.

62. The method of claim 54, wherein said disease includes cell proliferative disorders and/or differentiative disorders, such as carcinoma, sarcoma, solid tumours including prostate, breast, lung, testis, ovary, head and neck, brain or bone tumour, metastatic disorders or hematopoietic neoplastic disorders; disorders of bone metabolism, such as osteoporosis, osteodystrophy, osteomalacia, rickets, osteitis fibrosa cystica, renal osteodystrophy, osteosclerosis, osteopenia, fibrogenesis-imperfecta ossium, secondary hyperparathyrodism, hypoparathyroidism, hyperparathyroidism; periodontal disease, immune/autoimmune disorders such as, arthritis, multiple sclerosis, encephalomyelitis, myasthenia gravis, systemic lupus erythematosis, autoimmune thyroiditis, dermatitis, psoriasis, Sjogren's Syndrome, Crohn's disease, ulcer, iritis, conjunctivitis, ulcerative colitis, asthma, scleroderma, autoimmune uveitis, allergic encephalomyelitis, hearing loss, aplastic anemia, anemia, idiopathic thrombocytopenia, polychondritis, Graves' disease, graft-versus-host disease, allergy; hematopoietic disorders; cardiovascular disorders such as hypertension, atherosclerosis, congestive heart failure, coronary artery disease, arrhythmias, cardiomyopathies, artherosclerosis, hypertensive vascular disease, Raynaud disease, aneurysms, varicose veins, thrombophlebitis, phlebothrombosis, tumors, hemangionia, lymphangioma, glomus tumor (glomangioma), Kaposi sarcoma, angiosarcoma, hemangiopericytoma; blood clotting disorders, such as hemorrhagic diatheses, nonthrombocytopenic purpuras, thrombocytopenia, idiopathic thrombocytopenic purpura, HIV-associated thrombocytopenia, thrombotic microangiopathies, hemorrhagic diatheses, lymphomas, Hodgkin disease; liver disorders such as, cirrhosis, hepatocellular necrosis, liver fibrosis, Al-antitrypsin deficiency, hemochromatosis copper storage diseases and liver damage (especially drug or alcohol induced); bacterial, fungal or viral infection; pain or metabolic disorders, such as obesity, anorexia nervosa, cachexia, lipid disorders, and diabetes; AIDS and other pathological conditions in which serine proteases of the trypsin family are implicated.

63. The method of claim 54, wherein said disease is a disease in which trypsin proteins are implicated.

64. The method of claim 49, wherein said method of using a composition of matter comprises the method of monitoring the therapeutic treatment of a disease, comprising monitoring over a period of time the level of expression or activity of a polypeptide of claim 48, or the level of expression of a nucleic acid molecule of claim 48 in tissue from said patient, wherein altering said level of expression or activity over the period of time towards a control level is indicative of regression of said disease.

65. The method of claim 49, wherein said method of using a composition of matter comprises the method for identification of a compound that is effective in the treatment and/or diagnosis of a disease, comprising contacting a polypeptide of claim 48 or a nucleic acid molecule of claim 48 with one or more compounds suspected of possessing binding affinity for said polypeptide or nucleic acid molecule, and selecting a compound that binds specifically to said nucleic acid molecule or polypeptide.

66. The method of claim 49, wherein said method of using a composition of matter comprises the method for screening candidate compounds, comprising contacting a non-human transgenic animal of claim 48 with a candidate compound and determining the effect of the compound on the disease of the animal.

67. An isolated polypeptide comprising an amino acid sequence selected from the group consisting of SEQ ID NO:2, SEQ ID NO:4, SEQ ID NO:6, SEQ ID NO:8, SEQ ID NO:10, SEQ ID NO;12, SEQ ID NO;14, SEQ ID NO;16, SEQ ID NO:18, SEQ ID NO:20, SEQ ID NO:22, SEQ ID NO:24, and SEQ ID NO:26.

68. The isolated polypeptide of claim 67, wherein said polypeptide comprises the amino acid sequence recited in SEQ ID NO:22 or SEQ ID NO:26.

69. The isolated polypeptide of claim 67, wherein said polypeptide consists of an amino acid sequence selected from the group consisting of SEQ ID NO:2, SEQ ID NO:4, SEQ ID NO:6, SEQ ID NO:8, SEQ ID NO:10, SEQ ID NO;12, SEQ ID NO;14, SEQ ID NO;16, SEQ ID NO:18, SEQ ID NO:20, SEQ ID NO:22, SEQ ID NO:24, and SEQ ID NO:26.
